(19) United States
(12) Patent Application Publication
(10) Pub. No.: US 2025/0134507 A1
(43) Pub. Date: May 1, 2025

(54) OVER THE SCOPE CLIP

(71) Applicants: BOSTON SCIENTIFIC MEDICAL DEVICE LIMITED, Galway (IE); BOSTON SCIENTIFIC SCIMED, INC., Maple Grove, MN (US)

(72) Inventors: Rajivkumar SINGH, Pune (IN); Nachiket GOLE, Pune (IN); Deepak Kumar SHARMA, Muzaffarnagar (IN); Sharath Kumar G, Kanakapura (IN); Colby HARRIS, Norfolk, MA (US); Austin Grant JOHNSON, Worcester, MA (US)

(21) Appl. No.: 18/901,909

(22) Filed: Sep. 30, 2024

Related U.S. Application Data
(60) Provisional application No. 63/593,855, filed on Oct. 27, 2023.

(51) Int. Cl. A61B 17/00

(52) U.S. Cl. CPC A61B 17/0057; A61B 17/00234; A61B 2017/00296; A61B 2017/00367; A61B 2017/00477; A61B 2017/00584; A61B 2017/00619; A61B 2017/00862

(57) ABSTRACT

A system includes Adapter, Clip, Member, Wire and Mechanism. Adapter includes a proximal portion mounted over a distal end of an insertion device and a distal portion extending distally from the proximal portion. Clip includes Jaws to move between insertion configuration, in which Jaws extend about Adapter and are separated to receive tissue, and initial configuration, in which Clip is moved distally off of Adapter so that Jaws grip tissue. Member is releasably coupled to Clip and movably connected to Adapter. Member extends from a proximal portion to remain, in an operative configuration, outside body to a distal portion to be coupled to the distal end. Wire is received within Member and extends to a distal end releasably coupled to Clip via Link when predetermined force is applied. Mechanism selectively locks the distal portion of Member against proximal movement relative to Adapter during release of Link.

### PRIORITY CLAIM

The present disclosure claims priority to U.S. Provisional Patent Application Ser. No. 63/593,855 filed Oct. 27, 2023; the disclosure of which is incorporated herewith by reference.

### FIELD

The present disclosure relates to endoscopic devices and, in particular, relates to endoscopic clipping devices for treating tissue along the gastrointestinal tract.

### BACKGROUND

Physicians have become more willing to perform aggressive interventional and therapeutic endoscopic gastrointestinal (GI) procedures, which may increase the risk of perforating the wall of the GI tract or may require closure of the GI tract wall as part of the procedure. Such procedures may include, for example, the removal of large lesions, tunneling under the mucosal layer of the GI tract to treat issues below the mucosa, full thickness removal of tissue, treatment of issues on other organs by passing outside of the GI tract, and endoscopic treatment/repair of post-surgical issues (e.g., post-surgical leaks, breakdown of surgical staple lines, and anastomotic leaks).

Currently, tissue may be treated via endoscopic closure devices including through-the scope clips or over-the-scope clips. Over-the-scope clips may be particularly useful for achieving closure of larger tissue defects. These endoscopic closure devices can save costs for the hospital and may provide benefits for the patient. In some cases, however, current endoscopic closure devices may be difficult to use, time consuming to position, or insufficient for certain perforations, conditions and anatomies.

### SUMMARY

The present disclosure relates to a system for clipping tissue. The system includes an adapter including a proximal portion configured to be mounted over a distal end of an insertion device and a distal portion extending distally from the proximal portion. In addition, the system includes a clip configured to be mounted over the distal portion of the adapter. The clip includes first and second jaws configured to move between an insertion configuration, in which the first and second jaws extend about the adapter and are separated from one another to receive tissue therebetween, and an initial deployed configuration, in which the clip is moved distally off of the adapter so that the first and second jaws are drawn toward one another to grip tissue received therebetween.

Also, the system includes a first extending member releasably coupled to the clip and movably connected to the adapter. The first extending member extends from a proximal portion configured to remain, in an operative configuration, outside a patient's body to a distal portion configured to be coupled to a distal end of the insertion device. Furthermore, the system includes a first control wire slidably received within the first extending member and extending to a distal end releasably coupled to the clip via a first releasable link being configured to release when the first control wire is subject to a force exceeding a predetermined threshold value. In addition, the system includes a first locking mechanism configured to selectively lock the distal portion of the first extending member against proximal movement relative to the adapter during release of the first releasable link.

In an embodiment, the first extending member includes a recess and wherein the first locking mechanism includes a locking member configured to be into the recess of the first extending member when the first extending member is moved distally to a locking position.

In an embodiment, the first locking mechanism includes a first biasing member abutting the first locking member, the first biasing member being biased to urge the locking member against the first extending member so that, when the first extending member is moved to the locking position, the first biasing member pushes the first locking member into the recess of the first extending member.

In an embodiment, the first biasing member comprises an elastic member at least partially encircling the adapter and wherein the adapter includes a first opening through which the first extending member slidably extends.

In an embodiment, the first biasing member comprises a first leaf spring.

In an embodiment, the system further includes a first flexible member extending from a proximal end which, during use remains outside the patient's body accessible to a user to a distal end adjacent to the adapter. The first leaf spring extends from a proximal end coupled to the first flexible member to a distal end forming the locking member.

In an embodiment, the distal end of the first leaf spring is configured to lock the first flexible member against proximal movement relative to the adapter via frictional engagement with an outer surface of the first extending member.

In an embodiment, the system further includes a first tension member extending from a proximal end that, during use, remains outside the patient's body accessible to a user, to a distal end coupled to the first biasing member so that tension applied to the first tension member pulls the first biasing member out of engagement with the first extending member to release the first extending member for proximal movement relative to the adapter.

In an embodiment, the first locking mechanism includes a first locking member fixed to the adapter and a second locking member slidably mounted on the adapter, the first locking member defining a first lumen within which the first extending member is slidably received and the second locking member defining a second lumen within which the first extending member is slidably received, the first and second lumens being configured so that, when the second locking member is moved to a locking position, a portion of the first locking member received within the second lumen forces a portion of the second lumen to compress against the first extending member.

In an embodiment, the first locking member comprises a collet clevis fixed to the adapter and including a generally conic outer surface flaring radially outward from a reduced diameter distal end and wherein the second lumen tapers radially inward from an expanded diameter proximal end so that, the system further comprising a tension member coupled to the collet clevis so that the collet clevis can be drawn proximally over the collet forcing a radially inner surface of the collet clevis radially inward against the first extending member.

In an embodiment, the adapter includes a first tapered opening within which the first extending member is received, the first tapered opening having a diameter that gradually diminishes from a first diameter at a proximal end thereof to a reduced second diameter distal thereto, the first locking mechanism including a first compressible member fixed to the first extending member, the first compressible member being compressible to a reduced diameter configuration in which the first compressible member is substantially pressed against an outer surface of the first extendable member to an expanded configuration in which the first compressible member flares radially outward from a distal end fixed to the outer surface of the first extendable member to a radially expanded proximal end having a diameter greater than the reduced second diameter of the first tapered opening, the first compressible member being configured to compress as it passes through the first tapered opening and to expand to the expanded configuration under its bias when the proximal end of the first compressible member moves distally out of the first tapered opening so that contact between the radially expanded proximal end of the first compressible member and the adapter prevent the first compressible member from moving proximally into the first tapered opening.

In an embodiment, the adapter includes a second tapered opening within which the first extending member is received, the second tapered opening having a diameter that gradually diminishes from a first diameter at a distal portion thereof to a reduced second diameter proximal thereto, the first locking mechanism including a first gripping member slidably received within the second tapered opening, the first gripping member including a radially inner surface configured to grip the first extending member and being compressible to a reduced diameter configuration in which the first gripping member is substantially pressed against an outer surface of the first extendable member to lock a position of the first extendable member relative to the adapter and expandable to an expanded configuration in which the first gripping member flares radially outward to permit the first extendable member to slide therethrough.

In an embodiment, the first gripping member is coupled to a control member manipulable to move the first gripping member proximally within the second tapered opening forces the radially inner surface of the first gripping member against the first extendable member as the first gripping member is moved proximally into the second tapered opening.

In addition, the present disclosure relates to a method for treating tissue which includes inserting to a target area in a body lumen, an insertion device including a clip mounted over an adapter coupled to a distal end of the insertion device in an insertion configuration in which jaws of the clip are separated from one another to receive tissue therebetween; drawing a portion of tissue to be clipped into a channel of the adapter and between the jaws of the clip; moving the clip distally off of the adapter to permit the clip to move from the insertion configuration to an initial deployed configuration in which the jaws close over the portion of tissue by advancing distally a first extendable member slidably receiving a first control wire, wherein the first control wire is releasably coupled to the clip via a first releasable link; locking a distal portion of the first extendable member against proximal movement relative to the adapter; and pulling the first control wire distally against the first extendable member until a force exerted on the first releasable link until a coupling between the first control wire and the clip is released.

In an embodiment, the distal portion of the first extendable member is locked adjacent to the adapter.

In an embodiment, the first extendable member is slidably received within a first flexible member and wherein the first flexible member extends to a distal end coupled to the adapter, the distal portion of the first flexible member being locked distal to the distal end of the first flexible member.

In an embodiment, the first extendable member is locked against proximal movement relative to the adapter by a locking member that is inserted into a recess in an outer surface of the first extendable member.

In an embodiment, the locking member is coupled to a leaf spring that biases the locking member toward a gripping configuration in which a radially inner surface of the locking member is pressed against the outer surface of the first extendable member.

In an embodiment, the method further includes unlocking the first extendable member for proximal movement relative to the adapter after the first extendable member has been locked against proximal movement relative to the adapter by pulling a tension member proximally wherein the tension member is coupled to the leaf spring.

### BRIEF DESCRIPTION

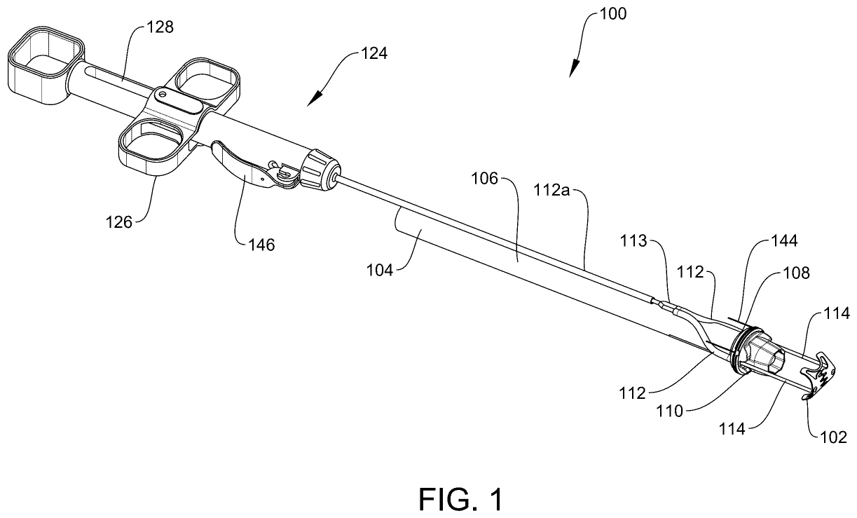

FIG. 1 shows a perspective view of a clipping system according to an exemplary embodiment of the present disclosure.

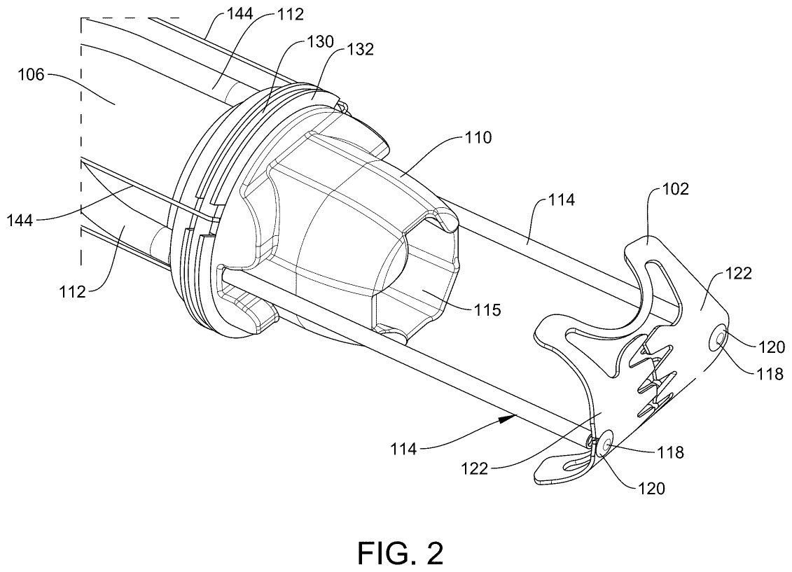

FIG. 2 shows a perspective view of the distal portion of the clipping system of FIG. 1, in a review configuration.

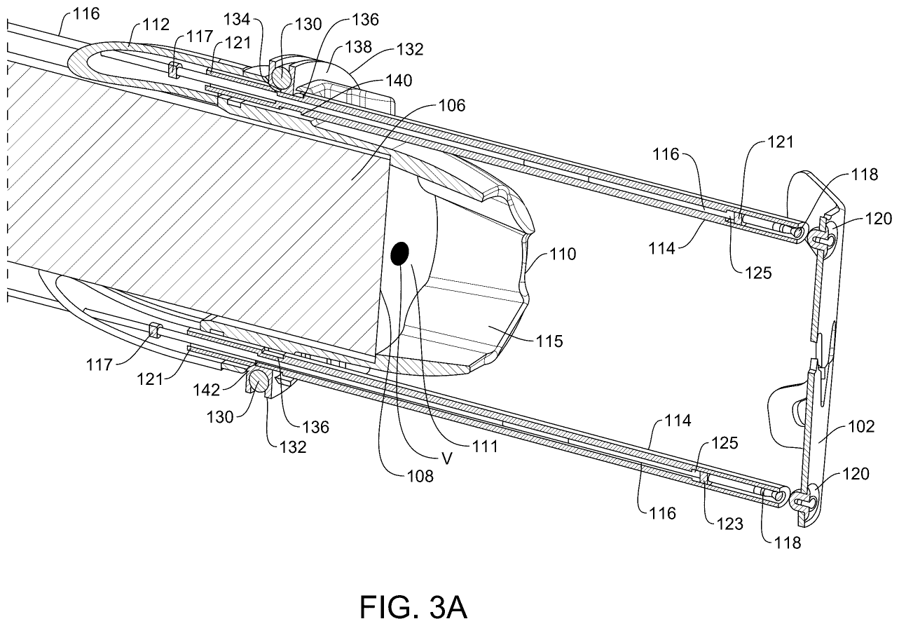

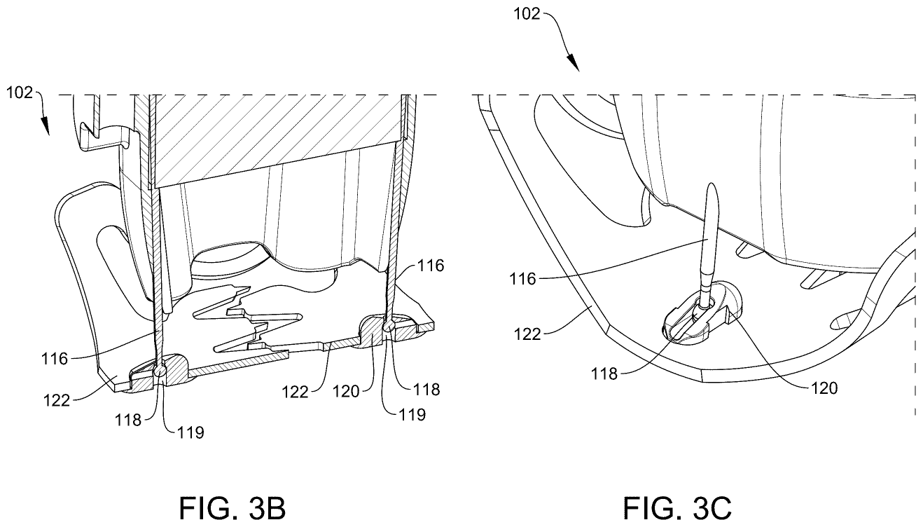

FIG. 3A shows a cross-sectional perspective view of the distal portion of the clipping system of FIG. 1, with the clip in the review configuration.

FIG. 3B shows a partially cross-sectional view of the distal portion of the clipping system of FIG. 1 including detail on a connection between the control wire and the clip.

FIG. 3C shows a partially cross-sectional view of the distal portion of the clipping system of FIG. 1 including detail on a connection between the control wire and the clip.

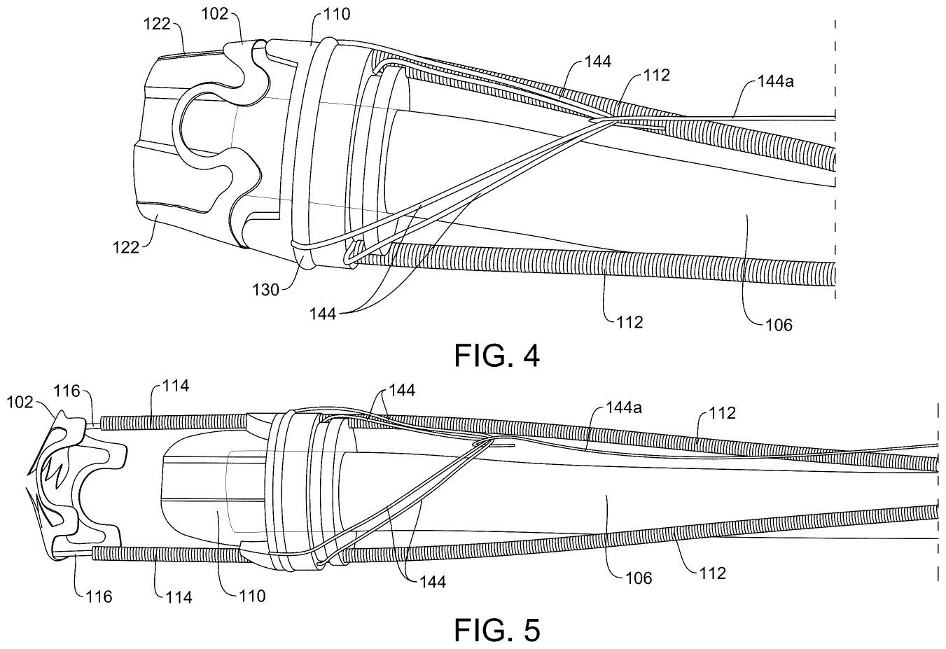

FIG. 4 shows a perspective view of the distal portion of the clipping system of FIG. 1 showing the pull wires and flexible tubes of the mechanism for deploying and locking a clip.

FIG. 5 shows a cross-sectional view of the distal portion of an adaptor of the system of FIG. 1.

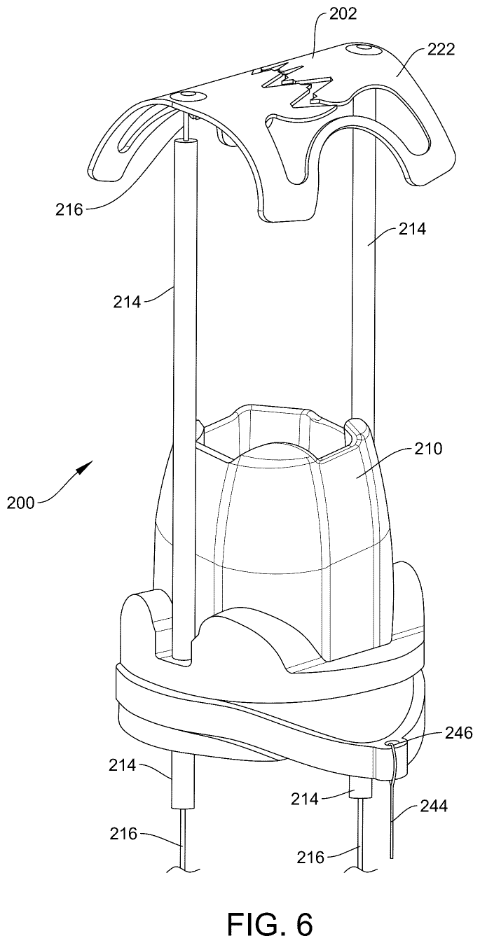

FIG. 6 shows a perspective view of the adaptor according to a second embodiment.

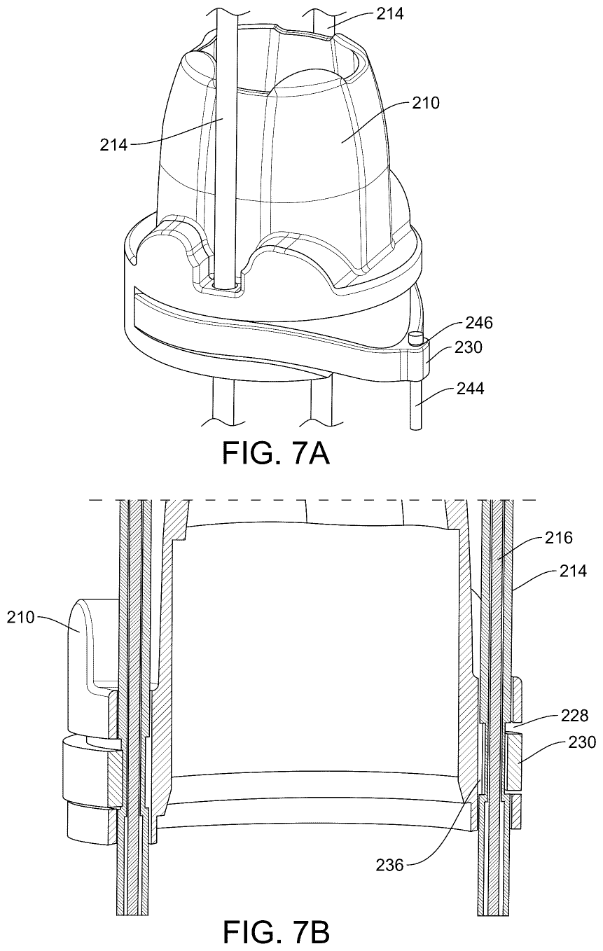

FIG. 7A shows a prospective view of the adaptor of FIG. 6.

FIG. 7B shows a cross-section view of the adapter of FIG. 6.

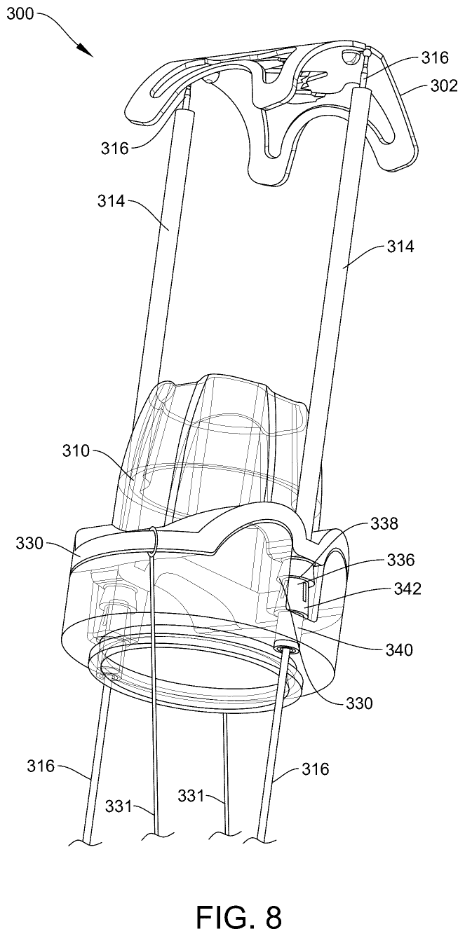

FIG. 8 shows a partially transparent view of an adaptor of a third embodiment.

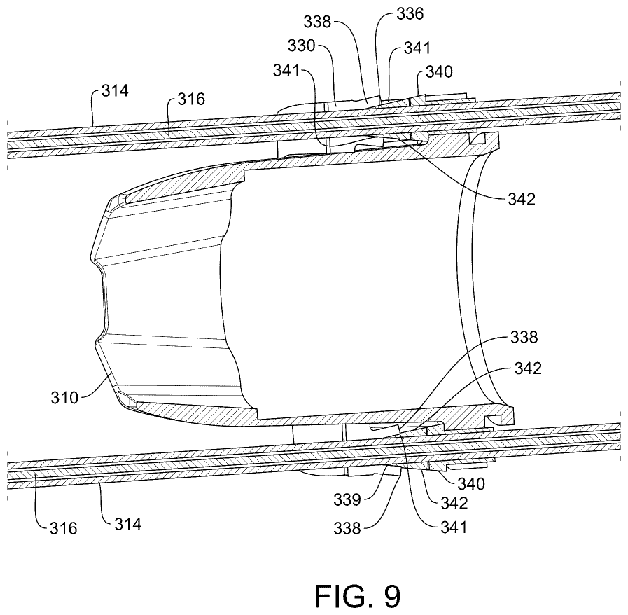

FIG. 9 shows a cross-section view of the adapter of FIG. 8.

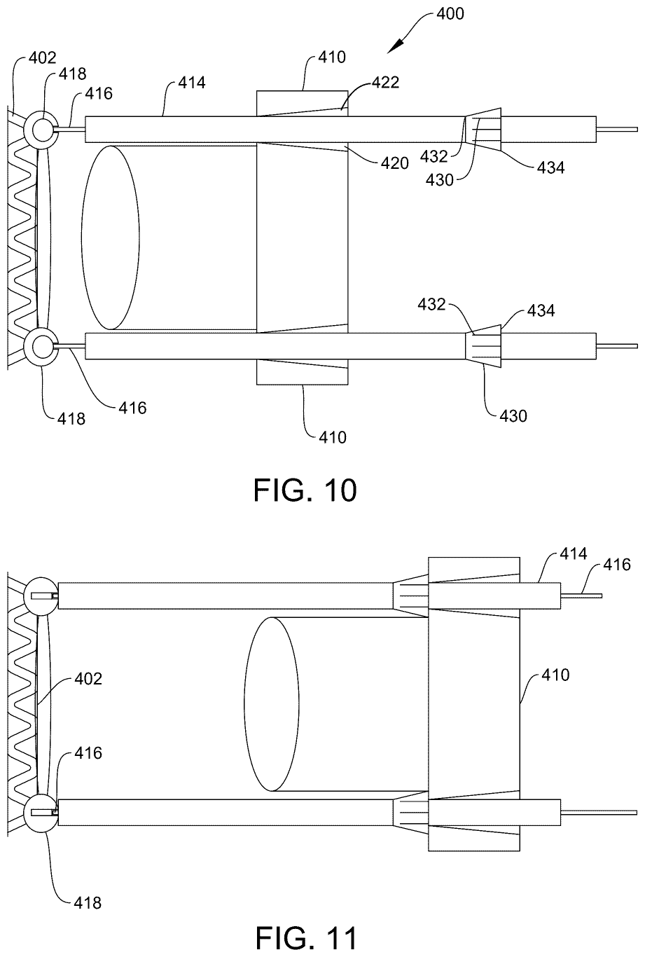

FIG. 10 shows a cross-sectional view of the distal end of a clipping system of a further embodiment in a first configuration.

FIG. 11 shows a cross-sectional view of the distal end of the clipping system of FIG. 10 in a second position.

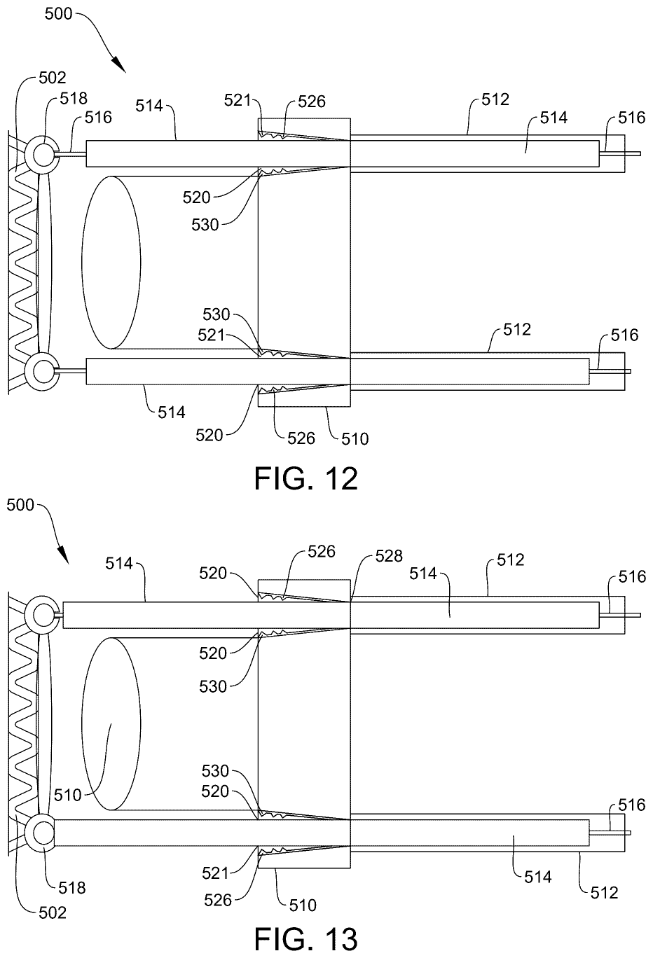

FIG. 12 shows a cross-sectional view of the distal end of a clipping system of a still further embodiment in a first configuration.

FIG. 13 shows a cross-sectional view of the distal end of the clipping system of FIG. 12 in a second position.

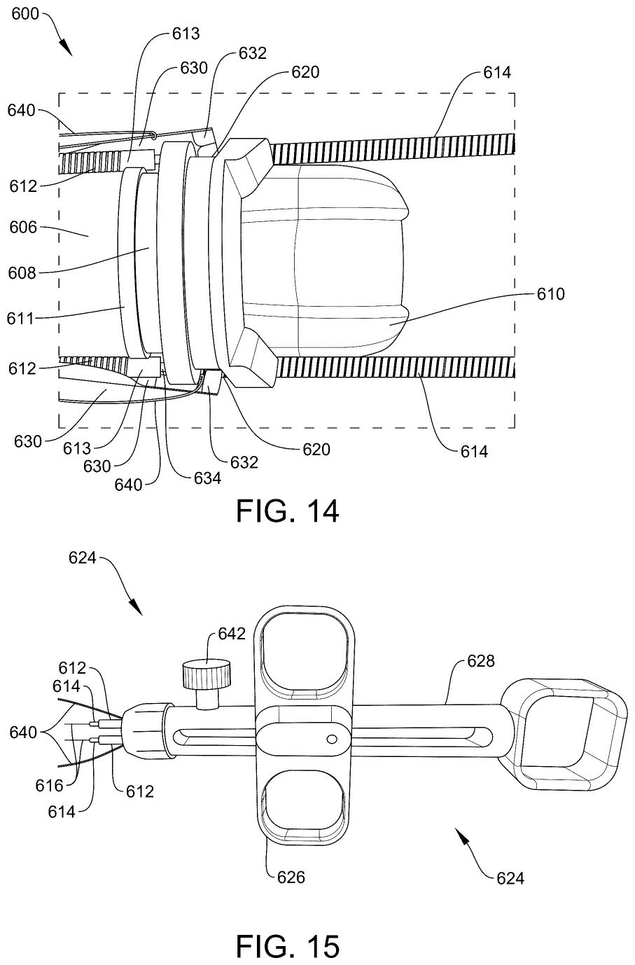

FIG. 14 shows a schematic representation of the locking mechanism of FIG. 10 in a fourth position.

FIG. 15 shows perspective view of the distal portion of a system according to an embodiment including a leaf spring of the mechanism for deploying and locking the clip.

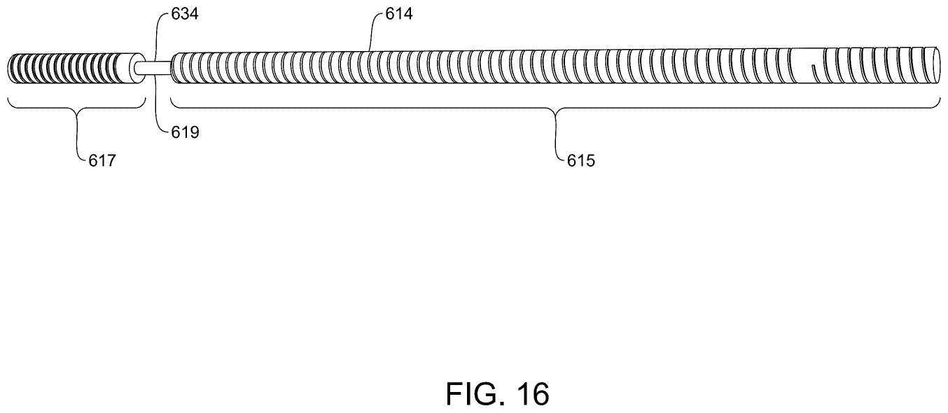

FIG. 16 shows a perspective view of a handle of the system of FIG. 14.

### DETAILED DESCRIPTION

The present disclosure may be further understood with reference to the following description and the appended drawings, wherein like elements are referred to with the same reference numerals. The present disclosure relates to a clipping system and, in particular, relates to an over-the-scope endoscopic clipping system, in which a clip is mounted on an adapter that is coupled to a distal end of an insertion device such as a flexible endoscope. The system includes a control wire that extends from a handle to the clip is slidably received in a first elongate member and the first elongate member is movable proximally and distally relative to the adapter to move the clip proximally and distally relative to the adapter to move the clip off of the adapter to clip tissue that had been drawn into the adapter.

The systems according to the disclosed embodiments includes various mechanisms for locking a position of the first elongate member at a distal location (e.g., at the adapter) so that, when it is desired to fully deploy the clip), a proximally directed tension force placed on the control wire pulls the clip against the distally locked first elongate member which applies an opposing distally directed force sufficient to sever a connection between the control wire and the clip. This is in contrast to prior over-the-scope clip systems in which this opposing force was applied by one or more elongate members that were locked at the handle (i.e., the proximal end of the system far from the clip). The locking of the elongate member(s) close to the clip enhances the function of the disclosed embodiments and permits the use of elongate members having column strength reduced relative to prior systems reducing material costs.

According to an exemplary embodiment, a distal end of a control wire in each of the extending members is coupled to the clip by a severable link such that, when a tension of a predetermined level is applied to the control wire, the control wire is separated from the clip. For example, a control wire extends through the first elongate member to extend through a first opening in the clip that is sized to prevent an enlarged end of the first control wire from passing proximally therethrough. Each control wire includes a frangible link between the distal end and a remaining length thereof that is configured to break, fail or otherwise separate when subject to a force exceeding a predetermined threshold force so that, during a final deployment of the clip, the frangible link breaks or separates to release the clip from control wires and the extending members.

After a user has positioned the clip as desired (e.g., after visually confirming that a target portion of tissue has been clipped by the clip as desired), the user may finally deploy the clip by separating the clip from the rest of the system. To do this, the user positions the distal end of the first elongate member against the proximal surface of the clip and locks a position of a distal portion of the first elongate member relative to the adapter and clip to prevent the clip from moving proximally. The user then applies proximally directed force to the control wire until the predetermined level is reached at which time the control wire is separated from the clip (e.g., by separating the enlarged distal end from the rest of the control wire, or by deforming or breaking the portion of the clip forming the opening through which the control wire passes so that the control wire is separated from the clip.

A system according to an exemplary embodiment includes first and second control wires a distal end of each of the control wires is connected to a corresponding end of the clip. The distal ends of the control wires are coupled to the clip so that, upon breaking or separating of the frangible link, the distal ends of the control wires remain attached to the clip or to the proximal portions of the respective control wires, preventing any shed parts during the final deployment, while the remaining lengths of the control wires may be removed from the body.

While the control wires remain coupled to the clip—e.g., the frangible link remains intact since a force applied thereon does not exceed the predetermined threshold value—the control wires may be moved longitudinally relative to the insertion device and the elongate members to move the clip between an insertion configuration in which the clip is stretched open over the adapter, an initial deployed configuration in which the clip is moved distally off of the adapter to clip tissue and a review configuration in which the adapter and the insertion device are moved proximally away from the clip to expand a field of view of the insertion device while the clip remains coupled to the proximal components of the system.

In the insertion configuration, the clip is mounted over the adapter in a proximal position maintained in the insertion configuration ready to receive tissue between jaws thereof while the clip's position minimizes its occlusion of the field of view of the endoscopic vision system. The insertion configuration is configured to facilitate insertion of the endoscope to a target site adjacent to tissue to be clipped while the system allows the clip to be deployed and clipped over tissue in an initial deployed configuration.

As the endoscope and the adapter are withdrawn proximally while the clip remains in place over the target tissue, the field of view of the vision system of the endoscope widens to show the clip and the tissue clipped thereby so that the operator can determine whether the position of the clip is desirable or in need of adjustment. If the operator determines that the clip is positioned as desired, the clip is deployed by releasing the clip from the control wires/extending members and left in place clipped over the target tissue. If the operator determines that the position of the clip needs adjustment, the endoscope and adapter are moved distally to a position adjacent to the clip. The clip is then drawn proximally over the adapter to reopen the clip which is drawn proximally off of the previously clipped tissue to move over the adapter forcing the clip to open against its natural bias as the clip slides proximally back over the adapter to return to the insertion configuration.

After the clip has been removed from the tissue and returned to the insertion configuration, the operator can re-position the endoscope as desired, draw a new portion of target tissue into the adapter (e.g., under suction or a grasper applied via a working channel of the endoscope) and once more deploy the clip from the adapter over the target tissue in the initial deployed position. The endoscope is then withdrawn proximally once again as the clip remains coupled to the device so that the device moves again into the review configuration.

The position of the clip and the clipped tissue are again observed and, this process may be repeated until the operator determines that the clip is positioned as desired. When the operator sees that the tissue over which the clip is closed is the desired portion of tissue, the frangible links of the control wires may be broken to release the clip therefrom, so that the clip moves to the final deployed configuration in which it is fully separated from the rest of the system and the insertion device. It will be understood by those of skill in the art that terms proximal and distal, as used herein, are intended to refer to a direction toward (proximal) and away from (distal), respectively, a user of the device.

As shown in FIGS. 1-5, a clipping system 100 for treating tissue defects and/or perforations according to an exemplary embodiment comprises a clip 102 configured to be inserted through, for example, a body lumen to a target area to clip a target tissue thereof. The clip 102 is insertable to the target area via an insertion device 104 including, e.g., an endoscope 106. As shown in FIGS. 1-5, the clip 102 is coupled to a distal end 108 of the endoscope 106 via an adapter 110, which is mounted over the distal end 108 of the endoscope 106.

The adapter 110 of this exemplary embodiment is sized and shaped to closely match a profile of the endoscope 106 and is hollow to define a central channel therein that is open to, for example, a working channel and a vision system V of the endoscope 106 (e.g., which terminate in a distal face 111 of the endoscope 106 as would be understood by those skilled in the art). The clip 102 is configured to be moved relative to the adapter 110 and the endoscope 106 via flexible members 112, each of which houses therein a tube 114 with a control wire 116 slidably received within each of the tubes 114.

Each of the control wires 116 includes a first crimp 117 located proximally of a proximal step 121 within a lumen of the corresponding tube 114 and a second crimp 123 located distally of a distal step 125 that is positioned within the lumen of the corresponding tube 114 so that movement of the control wires 116 distally within the tubes 114 pushes the first crimps 117 distally against the proximal steps 121 so that the tubes 114 move distally with the control wires 116.

When the position of the tubes 114 is not locked against proximal movement relative to the adapter 110 (to be described in more detail below), movement of the control wires 116 proximally within the tubes 114 draws the second crimps 123 proximally against the distal steps 125 pulling the tubes 114 proximally with the control wires 116. When the position of the tubes 114 is locked against proximal movement relative to the adapter 110, movement of the control wires 116 proximally within the tubes 114 draws the clip 102 proximally against the distal ends of the tubes 114 so that the column strength of the tubes 114 opposes the force applied to the control wires 116 and the control wires 116 are placed in tension. The clip 102 is releasably coupled to the control wires 116.

The clip 102 is movable from an insertion configuration to an initial deployed configuration and from the initial deployed configuration to a review configuration by moving the tubes 114 within the flexible members 112 as will be described in more detail below. The tubes 114 and the control wires 116 of the various embodiments herein are configured to permit withdrawal of the endoscope 106 by a range of 1 inch to 3 inches from the clip 102 when in the review configuration. As would be understood by those skilled in the art, a user may decide to withdraw the endoscope 106 by any distance less than the maximum range depending, for example, on constraints of the anatomy and/or the qualities of the vision system V of the endoscope 106 in regard to the distance required to provide the required view of the clip 102 and the surrounding area sufficient to permit the user to determine if the clip 102 has been deployed as desired.

If the user desires, the clip 102 may be moved from the review configuration back to the insertion configuration and then moved again to the initial deployed configuration in a new position. When the user is satisfied that the clip 102 has been positioned as desired, the clip 102 may be moved from the initial deployed configuration to a final deployed configuration in which the clip 102 is separated from the control wires 116 and the rest of the system 100 so that the clip 102 may be left in place clipped over desired tissue while the rest of the system 100 is withdrawn from the body.

As shown in FIGS. 2 and 3A-3C, each of the control wires 116 passes into an opening 119 in a mount 120 in a corresponding one of jaws 122 of the clip 102 includes an enlarged distal end 118 that resides, in an initial configuration (before final deployment of the clip 102), on a distal side of the opening 119. The opening 119 has a diameter selected to permit a proximal portion of the control wire 116 to pass therethrough but has a diameter smaller than a diameter of the enlarged distal end 118 so that each of the control wires 116 is fixed to the corresponding jaw 122. The flexible members 112 extend proximally from the adapter 110 to a junction 113 at which they merge to form a single flexible member 112a. The tubes 114 extend distally from a handle 124 at which they are slidably received while the control wires 116 extend through the tubes 114 to couple to a spool 126 that is slidably mounted on a body 128 of the handle 124. Movement of the spool 126 relative to the body 128 of the handle 124 moves the control wires 116 proximally and distally within the tubes 114 with interaction between the first and second crimps 117, 123, respectively, and the proximal and distal steps 121, 125, respectively, moves the tubes 114 proximally and distally along with the control wires 116.

In addition, the tubes 114 merge to form a single tube 114a proximally of the junction 113. As would be understood by those skilled in the art, the tube 114a splits into two tubes 114 at a location that is, when the clip 102 is in the insertion configuration, proximal of the junction 113 by a distance at least equal to the distance by which the clip 102 can be moved distally from the insertion configuration. That is, as would be understood by those skilled in the art, the tube 114a in a distal-most position, must remain proximal of the junction 113.

Those skilled in the art will understand that, alternatively, the jaws 122 may be formed with the openings 119 configured to yield when a proximally directed force of a predetermined magnitude is applied thereto via the control wires 116. In this case, the control wires 116 and the enlarged distal ends 118 would not separate from one another upon final deployment of the clip 102 but would be pulled intact out of the sockets.

As seen in FIGS. 2, 3A, and 3C, the adapter 110 includes a compressive ring 130 that resides in a slotted member 132 that extends circumferentially around at least a portion of the circumference of the adapter 110. The tubes 114 pass through the adapter 110 radially within the ring 130. The ring 130 extends around at least a portion of the circumference of the adapter 110 encompassing the locations at which the tubes 114 pass through the adapter 110. The slotted member 132 includes a radially inner projection 134 sized and shaped to be releasably received within corresponding recesses 136 formed in the tubes 114.

As would be understood by those skilled in the art and as will be described in more detail below, when it is desired to finally deploy the clip 102, the user operates the spool 126 to advance the tubes 114 distally until the recess 136 reaches a location radially within the slotted member 132. As this location is reached, the slotted member 132 is pushed radially inward by compressive force from the ring 130 so that the slotted member 132 enters the recesses 136. A distal surface 138 of the slotted member 132 of this embodiment extends substantially radially as does a distal surface 140 of each of the recesses 136 so that, when the slotted member 132 enters the recesses 136, the tubes 114 are locked preventing them from moving proximally relative to the slotted member 132.

For example, the recesses 136 are located so that they are separated from the distal ends of the tubes 114 by a distance selected to permit a more effective locking of the position of the distal ends of the tubes 114 (i.e., by positioning the recesses 136 close to the distal ends of the tubes 114 as opposed to locking the tubes 114 at their proximal ends (i.e., at the handle) the smaller portions of the tubes 114 extending distally from the recesses 136 will suffer less from bending/kinking when subject to compressive force. In an exemplary embodiment, the distance between each recess 136 and the distal end of the corresponding tube 114 may be between 12 and 75 mm.

A proximal surface 142 of a radially inner portion of the slotted member 132 according to this embodiment includes an optional curvature as locking of the tubes 114 against distal movement relative to the slotted member 132 is not required. The system 100 further includes a pair of tension members 144 (e.g., flexible threads or cables) that extend from distal ends coupled to the ring 130 (e.g., the tension members 144 may be wrapped around the ring 130). As shown in FIG. 2, the tension members 144 of this embodiment join to form a single tension member 144a that extends from a distal end coupled to the tension members 144 to a proximal end coupled to an actuator 146 on the handle 124.

As will be described in more detail below, when the tubes 114 have been locked by the slotted member 132 and the ring 130 and a user wishes to unlock the tubes 114 (e.g., if it is desired to open and re-position the clip 102), the actuator 146 may be operated to apply tension to the single tension member 144a and the tension members 144 so that the ring 130 is pulled proximally and moved out of the recesses 136. The tubes 114 may then be moved proximally relative to the slotted member 132 and the process of positioning the clip 102 and positioning it over a different portion of tissue may be repeated until the clip 102 has been positioned as desired.

When the user has positioned the clip 102 as desired (e.g., after the clip 102 has been initially deployed, observed in the review configuration, re-opened, re-positioned and clipped over a new portion of tissue), the user may finally deploy the clip 102. Specifically, the jaws 122 of the clip 102 of this embodiment are biased toward a closed tissue gripping configuration in which distal ends of the jaws 122 are drawn together to grip any tissue received therebetween. Thus, when the user advances the spool 126 distally relative to the body 128, the tubes 114 push the clip 102 distally until the jaws 122 move distally beyond a distal end of the adapter 110. The jaws 122 will then close under their natural bias and grip any tissue positioned therebetween.

As shown in FIG. 4 and as would be understood by those skilled in the art, before the clip 102 is moved distally off the adapter 110 (i.e., with the clip 102 in the insertion configuration), the clip 102 is held on the adapter 110 with the jaws 122 open so that a user may draw a target portion of tissue into the channel 115 of the adapter 110 (e.g., by applying suction or a grasper through a working channel of the endoscope 106). As shown in FIG. 2, in the insertion configuration, the clip 102 is mounted over the adapter 110 with jaws 122 separated from one another to receive tissue therebetween in an insertion configuration. To move the clip 102 from the insertion configuration to the initial deployed configuration (FIGS. 1-3 and 5), the tubes 114 are moved distally relative to the endoscope 106 (by operating the spool 126 as described above), moving the clip 102 distally until, as the jaws 122 project distally off the adapter 110, they close under their own bias (i.e., move to the initial deployed configuration) to clip the tissue that has been drawn into the channel 115 of the adapter 110.

After the jaws 122 have closed over this portion of tissue by moving the clip 102 to the initial deployed configuration, the clip 102 may be moved to a review configuration by extending the tubes 114 distally relative to the adapter 110 and moving the endoscope 106 and the adapter 110 proximally away from the clip 102 to expand the field of view of the vision system V as described above so that the clip 102 remains in place clipped over the tissue while the distal end of the endoscope 106 and the adapter 110 are spaced from the clip 102. In this configuration, the clip 102 remains tethered to the insertion device 104 via the control wires 116 while the field of view of the endoscope vision system relative to the clip 102 and the target tissue is widened while also allowing for movement of the endoscope 106 relative to the clip 102 to enable more extensive observation of the placement and/or position of the clip 102 relative to the target tissue.

As described below, if the user determines the position of the clip 102 is incorrect or sub-optimal, the user may move the endoscope 106 and the adapter 110 distally to a position adjacent to the clip 102 by sliding the endoscope 106 and the tubes 114 over the control wires 116 until the clip 102 abuts the distal end of the adapter 110. The endoscope 106 and the adapter 110 may be held in position immediately proximal of the clip 102 as the clip 102 is retracted proximally over the distal end of the adapter 110 by retracting the tubes 114 and the control wires 116 proximally. This forces the jaws 122 of the clip 102 open the previously clipped tissue (e.g., by, at the same time releasing any grasper or suction) and returns the clip 102 to the insertion configuration.

The user may then reposition the endoscope 106 and the clip 102 and repeat these steps to adjust the placement and/or position of the clip 102 relative to a target tissue as desired, prior to a final deployment of the clip 102 as will be described below. That is, if the operator determines in the review configuration that the clip 102 is not positioned as desired, the clip 102 may be reopened and removed from the tissue as many times as necessary so that the device can be repositioned until the clip 102 is closed as desired over the target portion of tissue.

When the user determines that the clip 102 has been positioned as desired clipped over the target portion of tissue, the user advances the tubes 114 distally until the recesses 136 align with the ring 130 and the radially inner projection 134 of the slotted member 132. The compression exerted by the ring 130 forces the radially inner projection 134 into the recesses 136 locking the position of the tubes 114 and preventing them from moving proximally relative to the adapter 110 as described above. The user then applies proximally directed force to the control wires 116 via the spool 126 until a predetermined tension is reached at which the connections between the control wires 116 and the jaws 122 is released.

For example, as described above, the enlarged distal ends 118 may, upon application of the predetermined tension, cause portions of the control wires 116 to fail separating proximal portions of the control wires 116 from the enlarged distal ends therefrom. This completely separates the clip 102 from the rest of the system 100 permitting the endoscope 106 along with the adapter 110, the proximal portions of the control wires 116 and the tubes 114, etc., to be withdrawn proximally from the patient's body leaving the clip 102 in position clipped over the target tissue.

If, for any reason, the user wishes to reposition the clip 102 after the tubes 114 have been locked in position by the slotted member 132, the system 100 further includes a mechanism for unlocking the connection between the tubes 114 and the slotted member 132. For example, as described above, the user may operate the tension members 144 to pull the ring 130 proximally removing the compressive force applied to the slotted member 132. This will release the tubes 114 for movement proximally and distally relative to the adapter 110 as desired until the user wishes to again lock the tubes 114 in position to finally deploy the clip 102. In addition, the user may apply tension to the tension members 144 after the clip 102 has been finally deployed so that the tubes 114 and the proximal portions of the control wires 116 may be withdrawn proximally into the flexible members 112 to prevent trauma to surrounding tissue as the endoscope 106 is withdrawn proximally from the patient's body.

A system 200 as shown in FIGS. 6, 7A and 7B is substantially similar in construction and operation to the system 100 except as described below. Specifically, the system 200 includes an adapter 210 configured to be mounted on an insertion device such as a flexible endoscope in the same manner described above in regard to the system 100 and with a similar handle and actuators configured to operate the various components in a like manner. However, the adapter 210 includes a clip ring 230 that is configured to interact with a recess 236 formed in each of the tubes 214 in a manner similar to the interaction between the recess 136 and the slotted member 132 described above. Specifically, when the recesses 236 are longitudinally aligned with the clip ring 230 the clip ring 230 compresses radially inward to enter the recesses 236 locking the position of the tubes 214 relative to the clip ring 230.

As seen in FIGS. 7A and 7B, the adapter 110 includes openings 228 through which a radially inner surface of the clip ring 230 can directly engage the tubes 214 and, when the tubes 214 are moved distally so that the recesses 236 align with the clip ring 230, the clip ring 230 squeezes into the recesses 236 to lock the position of the tubes 214 against proximal movement (relative to the adapter 210) in the same manner described above for the tubes 114 and the ring 130. That is, when the user of the system 200 wishes to finally deploy a clip 202 including a pair of jaws 222, the user advances the tubes 214 distally until the recesses 236 align with the clip ring 230.

At this point, the clip ring 230 moves radially inward into the recesses 236 to lock the tubes 214 against proximal movement. The user then applies tension to the control wires 216 until a predetermined tension is reached at which connections between the control wires 216 and the clip 202 are separated. Thus, the clip 202 is deployed in substantially the same manner as described above for the clip 102 (including the movement of the clip 202 between the insertion, initially deployed, review and finally deployed configurations described above).

The user may then unlock the tubes 214 from the clip ring 230 by applying tension to one or more tension member 244 to pull the clip ring 230 proximally out of engagement with the recesses 236. The tension member 244 of this embodiment is coupled to the clip ring 230 via an opening 246 in the clip ring 230. As would be understood by those skilled in the art, although the system 200 includes only a single tension member 244, any number of tension members may be employed to ensure that unlocking the tubes 214 from the clip ring 230 is achieved as desired. Similarly, the system 100 may include only a single tension member 144a or at least three tension members 144 as desired.

A system 300, as shown in FIGS. 8 and 9 is substantially similar in construction and operation to the systems 100 and 200 except as described below. Specifically, the system 300 includes an adapter 310 configured to be mounted on an insertion device such as a flexible endoscope in the same manner described above in regard to the systems 100, 200 and with a similar handle and actuators configured to operate the various components in a like manner. The system 300 includes a different mechanism for locking the tubes 314 relative to the adapter 310 as described below. As seen in FIGS. 8 and 9, the adapter 310 includes a collet clevis 330 that is slidably mounted on the adapter 310 and attached to one or more tension members 331 that extend back to a proximal end that remains, during use, outside the body accessible to a user of the system 300. Thus, when a user wishes to draw the collet clevis 330 proximally, the user simply pulls the tension member(s) 331 proximally drawing the collet clevis proximally as desired. Each of the tubes 314 passes slidably through a corresponding opening 336 in the collet clevis 330.

Each of the openings 336 is tapered from a maximum diameter at a proximal end 338 of the collet clevis 330 to a reduced diameter approximately equal to an outer diameter of the tubes 314 at a point 339 within the opening 336. A pair of collets 340 is fixed to the adapter 310 with each of the tubes 314 passing through a corresponding one of the collets 340. Thus, movement of the collet clevis 330 relative to the adapter 310 moves the collet clevis 330 distally and proximally over the tubes 314 relative to the collets 340. A proximal end of each of the openings 336 is configured to slidably receive a distal end 341 of a collet 340 that is fixed to the adapter 310 as the collet clevis 330 is moved proximally or distally relative to the adapter 310.

Each collet 340 includes ramped surfaces 342 that spread farther apart from one another at a proximal end of the collet 340 with a minimum diameter of each of the collets 340 at the distal ends 341. When the user wants to finally deploy the clip 302, the user pulls the collet clevis 330 proximally forcing each of the openings 336 proximally over the corresponding collet 340. As the collets 340 are inserted further distally into the tapered openings 336, the ramped surfaces 342 of the collets 340 press radially outward against the tapered surfaces of the openings 336 forcing the radially inner surfaces of the distal parts of the openings 336 (e.g., distal of the point 339) into contact with the tubes 314 until the tubes 314 are frictionally locked in place by the collet clevis 330.

The user then applies tension to the control wires 316 while maintaining the position of the tubes 314 until a predetermined tension is reached at which connections between the control wires 316 and the clip 302 are separated. Thus, the clip 302 is deployed in substantially the same manner as described above for the clip 102 (including the movement of the clip 302 between the insertion, initially deployed, review and finally deployed configurations described above). The user may then unlock the tubes 314 from the collet 340 by pushing the collet clevis distally so that the ramped surfaces 342 move out of the openings 336.

Those skilled in the art will understand that the various parts of the collet 340 and the collet clevis 330 can be rearranged in any number of ways to achieve the same locking effect. For example, the distal end of the collet 340 may be formed as a flared opening of a lumen that tapers radially inward toward a proximal end of the collet 340 while the collet clevis 330 includes a ramped outer surface flaring outward from a reduced diameter proximal end to an expanded distal portion. In this case, as the collet clevis 330 is pulled proximally over the collet 340, the decreasing diameter of the opening of the collet 340 will force the proximal end of the lumen of the collet clevis 330 closed against the tube 314.

A system 400, as shown in FIGS. 10 and 11 is substantially similar in construction and operation to the systems 100, 200 and 300 except as described below. Specifically, the system 400 includes an adapter 410 configured to be mounted on an insertion device such as a flexible endoscope in the same manner described above in regard to the systems 100, 200 and with a similar handle and actuators configured to operate the various components in a like manner. The system 400 includes a different mechanism for locking the tubes 414 relative to the adapter 410 as described below.

As seen in FIGS. 10 and 11, each of the tubes 414 includes an expandable member 430 fixed thereto. Each of the expandable members 430 includes a distal end 432 that has a diameter substantially equal to an outer diameter of the tube 414 to which it is attached and flares radially outward from this distal end 432 to an expanded proximal end 434. The expandable members are flexible so that they are compressible radially inward, and they are biased so that, when not acted on by an outside force, they flare outward into a generally conic shape with a maximum diameter of the expandible members at their proximal ends 434.

of the proximal ends 434 forms an abutting surface that resists distally directed forces applied thereto. The adapter 410 includes a pair of holes 420 extending therethrough with each of the holes 420 slidably receiving a corresponding one of the tubes 414 therein. The holes 420 are tapered so that a maximum diameter of each of the holes 420 is at a proximal end 422 thereof with the diameter of each of the holes 420 gradually diminishing through at least a portion of its length moving distally from its proximal end 422.

When the user of the system 400 wishes to finally deploy a clip 402 that has been clipped over target tissue as desired, the user advances the tubes 414 distally until each of the expandable members 430 enters the corresponding hole 420. As the user advances the tubes 414 further distally, the diminishing diameter of the taper of the holes 420 compresses the expandable members 430 until the expandable members 430 pass distally out of the holes 420.

When the proximal ends 434 of the expandable members 430 move distally out of the holes 420, the expandable members 430 flare outward under their natural bias with the abutting surfaces of the proximal ends 434 facing the distal end of the adapter 410. Thus, the tubes 414 are locked against proximal movement beyond the point at which the proximal ends 434 contact the distal end of the adapter 410. The user then applies tension to the control wires 416 until a predetermined tension is reached at which connections between the control wires 416 and the clip 402 are separated (e.g., by breaking the control wires 416 and leaving the enlarged distal ends 418 thereof attached to the clip 402).

Thus, the clip 402 is deployed in substantially the same manner as described above for the clips 102, 202 and 302 (including the movement of the clip 402 between the insertion, initially deployed, review and finally deployed configurations described above). The user may then withdraw the system 400 from the patient's body leaving the clip 402 in position clipped over the target tissue.

A system 500, as shown in FIGS. 12 and 13 is substantially similar in construction and operation to the systems 100, 200, 300 and 400 except as described below. Specifically, the system 500 includes an adapter 510 configured to be mounted on an insertion device such as a flexible endoscope in the same manner described above and with a similar handle and actuators configured to operate the various components in a like manner. The system 500 includes a different mechanism for locking the tubes 514 relative to the adapter 510 as described below. As seen in FIGS. 12 and 13, each of the tubes 514 is slidably received within holes 520 in the adapter 510.

Each of the tubes 514 is, in turn, slidably received within a corresponding one of a pair of flexible members 512. A distal end of each of the flexible members 512 is fixed to a compressible jaw 526 that is slidably received within a corresponding one of the holes 520. Thus, when the flexible members 512 are moved proximally relative to the adapter 510, the compressible jaws 526 are drawn proximally through the holes 520. As seen in FIGS. 12 and 13, each of the compressible jaws 526 includes a proximal end 528 that has a diameter substantially equal to or less than an inner diameter of a proximal end of the corresponding hole 520.

Each of the compressible jaws 526 flares radially outward from this proximal end 528 to an expanded distal end 530. The compressible jaws 526 are radially flexible so that they are compressible radially inward and they are biased so that, when not acted on by an outside force, they flare outward into a generally conic shape with a maximum diameter of each of the compressible jaws 526 at its proximal end 528. Each of the compressible jaws 526 includes a radially inner gripping surface that is configured to frictionally and/or mechanically engage an outer surface of the corresponding tube 514. Each of the holes 520 extends is tapered so that a maximum diameter of each of the holes 520 is at a distal end 521 thereof with the diameter of each of the holes 520 gradually diminishing through at least a portion of its length moving proximally from its distal end 521.

When the user of the system 500 wishes to finally deploy a clip 502 that has been clipped over target tissue as desired, the user draws the flexible members 512 proximally (e.g., using an actuator to which they are coupled on a handle similar to the handle 124) to draw each of the compressible jaws 526 proximally into the corresponding hole 520. As the user withdraws the flexible members 512 further proximally, the diminishing diameter of the taper of the holes 520 compresses the compressible jaws 526 until the gripping surfaces thereof engage the outer surfaces of the tubes 514, locking the tubes 414 against proximal movement.

The user then applies tension to the control wires 516 until a predetermined tension is reached at which connections between the control wires 516 and the clip 502 are separated (e.g., by breaking the control wires 516 and leaving the enlarged distal ends 518 thereof attached to the clip 502). Thus, the clip 502 is deployed in substantially the same manner as described above for the clips 102, 202, 302 and 402 (including the movement of the clip 502 between the insertion, initially deployed, review and finally deployed configurations described above). The user may then push the flexible members 512 distally to disengage the compressible jaws 526 from the tubes 514. The tubes 514 may then be withdrawn proximally into the adapter 510 and the flexible members 512 and the system 500 may be withdrawn from the patient's body leaving the clip 502 in position clipped over the target tissue.

As shown in FIGS. 14-16, a clipping system 600 for treating tissue defects and/or perforations according to an exemplary embodiment comprises a clip (not shown) of substantially the same design as in previous embodiments configured to be inserted through, for example, a body lumen to a target area to clip a target tissue thereof. The clip of the system 600 is insertable to the target area via an insertion device including, for example, an endoscope 606 in the same manner described above. The clip is coupled to a distal end 608 of the endoscope 606 via an adapter 610, which is mounted over the distal end 608 of the endoscope 606 (e.g., via a friction fit in the same manner as the adapters of the previously described embodiments).

The adapter 610 of this embodiment is sized and shaped to closely match a profile of the endoscope 606 and is hollow to define a central channel therein that is open to, for example, a working channel and a vision system of the endoscope 606 (e.g., which terminate in a distal face of the endoscope as described above). The clip is configured to be moved relative to the adapter 610 and the endoscope 606 via flexible members 612 each of which slidably houses therein a tube 614 with a control wire 616 slidably received within each of the tubes 614 in the same manner describe above for the previous embodiments.

The clip of this embodiment is similarly movable from an insertion configuration to an initial deployed configuration and from the initial deployed configuration to a review configuration by moving the tubes 614 within the flexible members 612. If the user desires, the clip of this embodiment may also be moved from the review configuration back to the insertion configuration and then moved again to the initial deployed configuration in a new position as described above. When the user is satisfied that the clip of this embodiment has been positioned as desired, the clip may be moved from the initial deployed configuration to a final deployed configuration in which the clip is separated from the control wires 616 and the rest of the system 600 so that the clip may be left in place clipped over desired tissue while the rest of the system 600 is withdrawn from the body.

As shown in FIG. 15, the flexible members 612 and the tubes 614 housed therein extend distally from a handle 624 at which they are coupled to a spool 626 that is slidably mounted on a body 628 of the handle 624. The control wires 616 of this embodiment may include, for example, the same or a similar mechanism including first and second crimps interfacing with proximal and distal steps within the lumens of the tubes 114 to ensure movement of the control wires relative to the adapter 610 generates a corresponding movement of the tubes 614 in the same manner described above in regard to the system 100.

As seen in FIG. 14, the flexible members 612 are configured, when the system 600 is assembled with an endoscope 606, to extend distally along an outer surface of the endoscope 606 to distal ends 613 that are adjacent to a proximal end 611 of the adapter 610. Each of the tubes 614 extends distally out of the distal end 613 of the corresponding flexible member 612 to pass through the adapter 610. The system 600 further includes a locking mechanism for selectively locking the tubes 614 against proximal movement (relative to the adapter 610 and the flexible members 612) that includes a pair of leaf springs 630 each of which is coupled to a distal end 613 of a corresponding one of the flexible members 612.

Each of the leaf springs 630 of this embodiment extends distally from a radially outer portion of the corresponding flexible member 612 to overlie a corresponding opening 620 in the adapter 610. Each of the leaf springs 630 includes a distal end 632 that projects through the corresponding opening 620 to engage the corresponding tube 614. Similar to the tubes 114 of the system 100 described above, each of the tubes 614 includes a recess 634 configured to lockingly engage the distal end 632 of the corresponding leaf spring 630 when the recess 634 is aligned with the corresponding opening 620.

That is, when the tubes 614 are advanced distally out of and retracted proximally into the flexible members 612, the natural bias of each leaf spring urges the distal end 632 thereof into contact with the corresponding tube 614 through the corresponding opening 620. Thus, as the tubes 614 are moved proximally and distally through the adapter 610, the distal ends 632 ride along the outside surfaces of the distal sections 617 of the tubes 614. When the user wants to finally deploy the clip (e.g., when the user has determined that the clip is clipped over target tissue as desired), the user advances the tubes 614 distally out of the flexible members 612 until the recesses 634 align with the openings 620. At this point, the bias of the leaf springs 630 forces the distal ends 632 thereof into the recesses 634 locking the tubes 614 against proximal movement relative to the adapter 610 and the tubes 614 in the same manner described above in regard to the tubes 114.

The user may then separate the clip of this embodiment from the control wires 616, the tubes 614 and the rest of the system 600 to finally deploy the clip in the same manner described above (e.g., by pulling proximally on the control wires 616 while the locked tubes 614 provide a distally directed force that eventually elevates a level of tension applied to a frangible link that separates the control wires from the clip). At this point the clip is completely separated from the rest of the system 600 and the user may withdraw the tubes 614 into the flexible members 612 for withdrawal of the system 600 from the patient's body by unlocking the tubes 614.

To do this, the user rotates a knob 642 on the handle 624 to draw a pair of tension members 640 proximally. Each of the tension members 640 of this embodiment extends distally from the handle 624 to loop around a corresponding one of the leaf springs 630. Thus, pulling the tension members 640 proximally pulls the leaf springs 630 so that the distal ends 632 are pulled out of engagement with the tubes 614 so that the tubes 614 may be drawn proximally into the flexible members 612.

As shown in more detail in FIG. 16, the tube 614 of this embodiment includes a proximal section 615 and a distal section 617 that are formed as coils of flexible material (e.g., stainless steel coils). The proximal section 615 of this embodiment is joined to the distal section 617 via a reduced diameter section 619 that is formed by a cylinder having an outer diameter less than the outer diameter of the proximal and distal sections 615, 617, respectively. Thus, the reduced diameter section 619 forms the recess 634 for each of the tubes 614. Those skilled in the art will understand that a similar construction may be used for the tubes of any of the above-described embodiments in which the tube includes a recess configured to engage a locking mechanism to lock a position of the tube relative to the adapter and/or the flexible member.

It will be apparent to those skilled in the art that various modifications may be made in the present disclosure, without departing from the scope of the disclosure. Furthermore, those skilled in the art will understand that the features of any of the various embodiments may be combined in any manner that is not inconsistent with the description and/or the functionality of the embodiments.

1-13. (canceled)

14. A system for clipping tissue, comprising:
an adapter including a proximal portion configured to be mounted over a distal end of an insertion device and a distal portion extending distally from the proximal portion;
a clip configured to be mounted over the distal portion of the adapter, the clip including first and second jaws configured to move between an insertion configuration, in which the first and second jaws extend about the adapter and are separated from one another to receive tissue therebetween, and an initial deployed configuration, in which the clip is moved distally off of the adapter so that the first and second jaws are drawn toward one another to grip tissue received therebetween;
a first extending member releasably coupled to the clip and movably connected to the adapter, the first extending member extending from a proximal portion configured to remain, in an operative configuration, outside a patient's body to a distal portion configured to be coupled to a distal end of the insertion device;
a first control wire slidably received within the first extending member and extending to a distal end releasably coupled to the clip via a first releasable link being configured to release when the first control wire is subject to a force exceeding a predetermined threshold value; and
a first locking mechanism configured to selectively lock the distal portion of the first extending member against proximal movement relative to the adapter during release of the first releasable link.

15. The system of claim 14, wherein the first extending member includes a recess and wherein the first locking mechanism includes a locking member configured to be into the recess of the first extending member when the first extending member is moved distally to a locking position.

16. The system of claim 15, wherein the first locking mechanism includes a first biasing member abutting the first locking member, the first biasing member being biased to urge the locking member against the first extending member so that, when the first extending member is moved to the locking position, the first biasing member pushes the first locking member into the recess of the first extending member.

17. The system of claim 16, wherein the first biasing member comprises an elastic member at least partially encircling the adapter and wherein the adapter includes a first opening through which the first extending member slidably extends.

18. The system of claim 16, wherein the first biasing member comprises a first leaf spring.

19. The system of claim 18, further comprising:
a first flexible member extending from a proximal end which, during use remains outside the patient's body accessible to a user to a distal end adjacent to the adapter,
wherein the first leaf spring extends from a proximal end coupled to the first flexible member to a distal end forming the locking member.

20. The system of claim 19, wherein the distal end of the first leaf spring is configured to lock the first flexible member against proximal movement relative to the adapter via frictional engagement with an outer surface of the first extending member.

21. The system of claim 16, further comprising:
a first tension member extending from a proximal end that, during use, remains outside the patient's body accessible to a user, to a distal end coupled to the first biasing member so that tension applied to the first tension member pulls the first biasing member out of engagement with the first extending member to release the first extending member for proximal movement relative to the adapter.

22. The system of claim 14, wherein the first locking mechanism includes a first locking member fixed to the adapter and a second locking member slidably mounted on the adapter, the first locking member defining a first lumen within which the first extending member is slidably received and the second locking member defining a second lumen within which the first extending member is slidably received, the first and second lumens being configured so that, when the second locking member is moved to a locking position, a portion of the first locking member received within the second lumen forces a portion of the second lumen to compress against the first extending member.

23. The system of claim 22, wherein the first locking member comprises a collet clevis fixed to the adapter and including a generally conic outer surface flaring radially outward from a reduced diameter distal end and wherein the second lumen tapers radially inward from an expanded diameter proximal end so that, the system further comprising a tension member coupled to the collet clevis so that the collet clevis can be drawn proximally over a collet forcing a radially inner surface of the collet clevis radially inward against the first extending member.

24. The system of claim 14, wherein the adapter includes a first tapered opening within which the first extending member is received, the first tapered opening having a diameter that gradually diminishes from a first diameter at a proximal end thereof to a reduced second diameter distal thereto, the first locking mechanism including a first compressible member fixed to the first extending member, the first compressible member being compressible to a reduced diameter configuration in which the first compressible member is substantially pressed against an outer surface of the first extendable member to an expanded configuration in which the first compressible member flares radially outward from a distal end fixed to the outer surface of the first extendable member to a radially expanded proximal end having a diameter greater than the reduced second diameter of the first tapered opening, the first compressible member being configured to compress as it passes through the first tapered opening and to expand to the expanded configuration under its bias when the proximal end of the first compressible member moves distally out of the first tapered opening so that contact between the radially expanded proximal end of the first compressible member and the adapter prevent the first compressible member from moving proximally into the first tapered opening.

25. The system of claim 14, wherein the adapter includes a second tapered opening within which the first extending member is received, the second tapered opening having a diameter that gradually diminishes from a first diameter at a distal portion thereof to a reduced second diameter proximal thereto, the first locking mechanism including a first gripping member slidably received within the second tapered opening, the first gripping member including a radially inner surface configured to grip the first extending member and being compressible to a reduced diameter configuration in which the first gripping member is substantially pressed against an outer surface of the first extendable member to lock a position of the first extendable member relative to the adapter and expandable to an expanded configuration in which the first gripping member flares radially outward to permit the first extendable member to slide therethrough.

26. The system of claim 25, wherein the first gripping member is coupled to a control member manipulable to move the first gripping member proximally within the second tapered opening forces the radially inner surface of the first gripping member against the first extendable member as the first gripping member is moved proximally into the second tapered opening.

27. A method for treating tissue, comprising:
inserting to a target area in a body lumen, an insertion device including a clip mounted over an adapter coupled to a distal end of the insertion device in an insertion configuration in which jaws of the clip are separated from one another to receive tissue therebetween;
drawing a portion of tissue to be clipped into a channel of the adapter and between the jaws of the clip;
moving the clip distally off of the adapter to permit the clip to move from the insertion configuration to an initial deployed configuration in which the jaws close over the portion of tissue by advancing distally a first extendable member slidably receiving a first control wire, wherein the first control wire is releasably coupled to the clip via a first releasable link;
locking a distal portion of the first extendable member against proximal movement relative to the adapter; and
pulling the first control wire distally against the first extendable member until a force exerted on the first releasable link until a coupling between the first control wire and the clip is released.

28. The method of claim 27, wherein the distal portion of the first extendable member is locked adjacent to the adapter.

29. The method of claim 28, wherein the first extendable member is slidably received within a first flexible member and wherein the first flexible member extends to a distal end coupled to the adapter, the distal portion of the first flexible member being locked distal to the distal end of the first flexible member.

30. The method of claim 29, wherein the first extendable member is locked against proximal movement relative to the adapter by a locking member that is inserted into a recess in an outer surface of the first extendable member.

31. The method of claim 30, wherein the locking member is coupled to a leaf spring that biases the locking member toward a gripping configuration in which a radially inner surface of the locking member is pressed against the outer surface of the first extendable member.

32. The method of claim 31, further comprising unlocking the first extendable member for proximal movement relative to the adapter after the first extendable member has been locked against proximal movement relative to the adapter by pulling a tension member proximally wherein the tension member is coupled to the leaf spring.
